(19) United States
(12) Patent Application Publication
(10) Pub. No.: US 2005/0255063 A1
(43) Pub. Date: Nov. 17, 2005

(54) Candy cosmetics

(76) Inventor: Jan L. Fenwick-Le Vine, Chatsworth, CA (US)

Correspondence Address: Jan Fenwick Le Vine, 21618 Los Alimos St, Chatsworth, CA, 91311, US

(21) Appl. No.: 10/845,217

(22) Filed: May 14, 2004

(51) Int. Cl. A61K 7/021; A61K 9/68

(52) U.S. Cl. 424/63; 424/440

(57) ABSTRACT

A cosmetic product comprising a sweetener.

### BACKGROUND

1. Field

This invention relates to candy cosmetics.

2. Relevant Art

U.S. Pat. No. 6,528,071 discloses cosmetic compositions, comprising: a.) from about 0.01% to about 50%, by weight, of vitamin B.sub.3 compound; b.) from about 0% to about 90%, by weight, of an emollient component comprising from 0% to about 100%, by weight, of an oil liquid at ambient temperature; c.) from about 0.01% to about 40%, by weight, of a polar solvent; d.) from about 0% to about 90%, by weight, of a solidifying agent; and e.) from about 0% to about 90%, on an anhydrous basis, of a color wherein the vitamin B.sub.3 compound is added to the composition such that the concentration of the vitamin B.sub.3 compound exceeds the saturation solubility of the vitamin B.sub.3 compound in the polar solvent. U.S. Pat. No. 6,528,071 is herein incorporated by reference in its entirety.

U.S. Pat. No. 6,524,598 discloses cosmetic compositions comprising a combination of non-emulsifying and emulsifying crosslinked siloxane elastomers. U.S. Pat. No. 6,524,598 is herein incorporated by reference in its entirety.

U.S. Pat. No. 6,509,023 discloses a cosmetic composition comprising a specific functionalized organopolysiloxane which is linear or cyclic. Said functionalized organopolysiloxane is used as emollient and/or carrier and/or anti-transfer agent in cosmetic compositions. U.S. Pat. No. 6,509,023 is herein incorporated by reference in its entirety.

U.S. Pat. No. 6,479,040 discloses a cosmetic formulation. The cosmetic formulation comprises a plurality of spheres having a first desiccated volume and having a second hydrated volume. The hydrated volume is greater than the desiccated volume. U.S. Pat. No. 6,479,040 is herein incorporated by reference in its entirety.

U.S. Pat. No. 6,406,685 discloses a cosmetic composition comprising, in a cosmetically acceptable medium, at least one compound of formula (I) in which R.sub.1, and R′.sub.1 are chosen from alkyl radicals and R.sub.2, R′.sub.2, R.sub.3, R′.sub.3, R.sub.4, R′.sub.4, R.sub.5, and R′.sub.5 are chosen from a hydrogen atom, a halogenated radical, a hydroxyl radical, an alkyl, alkyloxy, acyl, or acyloxy radical having 1 to 6 carbon atoms. The use of compounds of formula (I) for coating substrate particles, the resulting powder substance, and its use in a cosmetic composition. U.S. Pat. No. 6,406,685 is herein incorporated by reference in its entirety.

U.S. Pat. No. 6,395,262 discloses a cosmetic composition, which may be in the form of an emulsion, the composition comprising a specific copolymer comprised of monoolefinically unsaturated C.sub.3-C.sub.6 carboxylic acid monomer and fatty-chain monomeric ester of acrylic acid. The disclosure also relates to the use of the said copolymer in cosmetics, in particular for improving the staying power of the composition. U.S. Pat. No. 6,395,262 is herein incorporated by reference in its entirety.

U.S. Pat. No. 6,379,729 discloses a water-soluble color-stabilized food containing ascorbic acid or its salt and at least one water-soluble color selected from the group consisting of coal-tar color, anthocyanin color and carthamus yellow, said food further containing sorbic acid and/or its salt and/or a salt of sulfurous acid so that the fading of the water-soluble color can be prevented and the stability thereof can be increased. U.S. Pat. No. 6,379,729 is herein incorporated by reference in its entirety.

U.S. Pat. No. 6,224,888 discloses cosmetic compositions, comprising: a.) from about 0.01% to about 50%, by weight, of vitamin B.sub.3 compound; b.) from about 0% to about 90%, by weight, of an emollient component comprising from 0% to about 100%, by weight, of an oil liquid at ambient temperature; c.) from about 0.01% to about 40%, by weight, of a polar solvent; d.) from about 0% to about 90%, by weight, of a solidifying agent; and e.) from about 0% to about 90%, on an anhydrous basis, of a color wherein the vitamin B.sub.3 compound is added to the composition such that the concentration of the vitamin B.sub.3 compound exceeds the saturation solubility of the vitamin B.sub.3 compound in the polar solvent. U.S. Pat. No. 6,224,888 is herein incorporated by reference in its entirety.

U.S. Pat. No. 6,197,318 discloses a composition for external use which contains xyloglucan. It can also contain an ultraviolet shielding agent, a thickening polysaccharide, a thickening polysaccharide and sericin, a carboxyvinylpolymer, or an alkyl-modified carboxyvinylpolymer. U.S. Pat. No. 6,197,318 is herein incorporated by reference in its entirety.

### SUMMARY

A candy cosmetic that includes a sweetener. In one embodiment, there is disclosed a lipstick that includes a sweetener. In another embodiment, there is disclosed a lip gloss that includes a sweetener. In another embodiment, there is disclosed a lip balm that includes a sweetener. In another embodiment, there is disclosed a nail polish that includes a sweetener. In another embodiment, there is disclosed a flavored whipped cream which may be used as a shaving cream toy.

### DETAILED DESCRIPTION OF THE INVENTION

In one embodiment, there is disclosed a lip gloss that includes a sweetener. In another embodiment, the lip gloss can also include an oil. In another embodiment, the lip gloss can also include a fat and oil mixture. In another embodiment, the lip gloss can also include an emulsifier. In another embodiment, the lip gloss can also include a filler. In another embodiment, the lip gloss can also include flavors and/or colorings.

In one embodiment, there is disclosed a lipstick that includes a sweetener. In another embodiment, the lipstick can also include a moisturizer. In another embodiment, the lipstick can also include a fat. In another embodiment, the lipstick can also include a fat and oil mixture. In another embodiment, the lipstick can also include a filler.

In another embodiment, there is disclosed a lip balm that includes a sweetener. In another embodiment, the lip balm can also include a moisturizer. In another embodiment, the lip balm can also include a fat. In another embodiment, the lip balm can also include a fat and oil mixture. In another embodiment, the lip balm can also include a filler. In another embodiment, the lip balm include also an emulsifier.

In another embodiment, there is disclosed a nail polish that includes a sweetener. In another embodiment, the nail polish also includes a solvent. In another embodiment, the nail polish also includes an edible shellac. In another embodiment, the nail polish also includes colorings and/or flavorings.

In one embodiment, suitable sweeteners include sugars, for example, monosaccharides, disaccharides, and/or polysaccharide sugars, and/or mixtures of two or more sugars. In another embodiment, sweeteners include one or more of the following: sucrose or table sugar; honey or a mixture of low molecular weight sugars not including sucrose; glucose or grape sugar or corn sugar; molasses; dextrose; corn sweetener; corn syrup or glucose syrup; fructose or fruit sugar; lactose or milk sugar; maltose or malt sugar or maltobiose; sorghum syrup; mannitol or manna sugar; sorbitol or d-sorbite or d-sobitol; fruit juice concentrate; and/or mixtures or blends of one or more of these ingredients.

In another embodiment, suitable sweeteners include artificial sweeteners, non-nutritive sweeteners, and/or non-caloric sweeteners. Suitable artificial sweeteners include: saccharin or Sweet N Low®; Aspartame or Equal® or NutraSweet®; sucralose or Splenda®; acesulfame K or acesulfame potassium or Sunette®; and/or alitame available from Pfizer.

In another embodiment, suitable sweeteners include mixtures of one or more sugars and/or one or more artificial sweeteners.

In one embodiment, suitable fats are triglycerides, or are mixtures of triglycerides having a melting point greater than about 70° F. In another embodiment, oils are triglycerides, or are mixtures of triglycerides having a melting point less than about 70° F.

In one embodiment, suitable fats include vegetable shortening; partially hydrogenated vegetable shortening; lard; partially saturated vegetable shortening; margarines; butter; shortening; partially hydrogenated and/or partially saturated food oils containing ester groups having a melting point greater than about 70° F. In another embodiment, suitable fats include a partially or fully hydrogenated and/or a partially or fully saturated oil that has a melting point of at least about 70° F., suitable oils to hydrogenate and/or saturate include cannola oil, grape seed oil, coconut oil, corn oil, cotton seed oil, grape seed oil, olive oil, peanut oil, safflower oil, sesame oil, soy bean oil, sunflower oil, walnut oil, and/or mixtures of two or more of these oils. In another embodiment, suitable fats include animal fat. In another embodiment, suitable fats include mixtures of two or more of the fats mentioned above emulsifiers.

In another embodiment, suitable fats include fats and/or fat mixtures having a melting point greater than about 95° F. In another embodiment, suitable fats include fats and/or fat mixtures having a melting point greater than about 105° F. In another embodiment, suitable fats include fats and/or fat mixtures having a melting point greater than about 115° F.

One suitable oil is 100% pure corn oil commercially available from Vons.

In another embodiment, suitable oils include cannola oil, grape seed oil, coconut oil, corn oil, cotton seed oil, grape seed oil, olive oil, peanut oil, safflower oil, sesame oil, soy bean oil, sunflower oil, walnut oil, almond oil, hazel nut, and/or mixtures of two or more of these oils, where the oil or oil mixture has a melting point less than about 70° F.

In another embodiment, suitable oils include cannola oil, grape seed oil, coconut oil, corn oil, cotton seed oil, grape seed oil, olive oil, peanut oil, safflower oil, sesame oil, soy bean oil, sunflower oil, walnut oil, almond oil, hazel nut, and/or mixtures of two or more of these oils, where the oil or oil mixture has a melting point less than about 70° F.

In another embodiment, suitable emulsifiers include lecithin, surface-active agents, phospholipids, phosphoglycerides, protective colloids, protein polymers, carbohydrate polymers, long chain alcohols and fatty acids, and mixtures or two or more of the above.

In another embodiment, suitable fillers include inulin, alantin, maltodextrin, non-sweet complex carbohydrates, cyclodextrin, dextrose, corn syrup solids, corn starch, flour, sweet whey protein, non-fat dry milk, whole dry milk, and mixtures of two or more of the above fillers.

In another embodiment, suitable colors include FLAMING RED, FD&C red #40 lake; CHERRY PINK; FD&C red #4 Lake, and/or VIOLET, FD&C Blue #1 Lake; commercially available from Linnea's, Inc. of Akron, Ohio and San Bernardino, Calif.

In another embodiment, suitable edible shellac is confectioners glaze 78-8200 commercially available from CK Products of Fort Wayne, Ind.

A suitable sweetener includes confectioner's sugar having a mixture of sugar and corn starch commercially available from C&H Sugar Co., Inc. of Crockett, Calif.

A suitable fat is Crisco, commercially available from JM Smucker Co. of Orrville, Ohio.

A suitable emulsifier is lecithin 8-198 commercially available from Linnea's of Kent, Ohio.

A suitable flavor is cotton candy flvart os: #02-CF 119, and watermelon flvart os #02CF118, commercially available from Western Flavors and Fragrances of Livermire, Calif.

In another embodiment, a suitable filler is inulin iq commercially available as Frutafit® from Imperial Suiker Unie of Sugarland, Tex.

In another embodiment, suitable moisturizers include beeswax, petrolatum, petroleum jelly, paraffin, lanolin, mineral oil, and/or mixtures of two or more of the above moisturizers.

In another embodiment, suitable solvents include water, ethanol, and mixtures of water and ethanol.

In another embodiment, there is disclosed a flavored whipped cream which may be used as a shaving cream toy.

### EXAMPLES

Lip Gloss

10 g powdered confectioners sugar (may use 0 g-25 g)
20 g of oil and fat mixture including 13 g Crisco and 7 g corn oil, 1/10 g Lecithin, 10 g Inulin
   Flavors and Colorings
   0.2 mL cotton candy flvart os #02-CF1119
   20 drops FD&C red #40 food coloring
      The Crisco and corn oil were melted and blended together, then the remaining ingredients were added and mixed and the mixture was ground for the best texture.
      Lipstick
10% beeswax
10 g powdered confectioners sugar
15 g commercial shortening having a melting point of at least 110° F.
0-3 g corn oil
at least 10 g of inulin
   The commercial shortening and corn oil were melted and mixed together, then the beeswax was added, followed by the dry ingredients. The mixture was then molded to form a lipstick.
   Nail Polish
125 g powdered confections sugar
25 g water
5 g commercial edible shellac
coloring
flavoring
   The water was mixed with the powdered confectioners sugar and coloring and flavoring, then the commercial edible shellac was added, all at room temperature to form a candy nail polish.

1. A cosmetic product comprising a sweetener.

2. The cosmetic product of claim 1, wherein the sweetener comprises confectioners sugar.

3. The cosmetic of claim 1, wherein the cosmetic is edible.

4. The cosmetic of claim 1, further comprising an oil.

5. The cosmetic product of claim 1, further comprising a fat.

6. The cosmetic product of claim 1, further comprising a fat and oil mixture.

7. The cosmetic product of claim 1, further comprising an emulsifier.

8. The cosmetic product of claim 1, further comprising a filler.

9A. The cosmetic product of claim 1, further comprising at least one flavoring.

9B. The cosmetic product of claim 1, further comprising salt.

10. The cosmetic product of claim 1, further comprising at least one coloring.

11. A cosmetic product further comprising a moisturizer.

12. The cosmetic product of claim 1, further comprising a solvent.

13. An edible candy lip gloss comprising a sweetener, an oil, an emulsifier, a flavoring, and a coloring.

14. The lip gloss of claim 13, further comprising a fat.

15. The lip gloss of claim 13, further comprising a filler.

16. An edible candy lipstick comprising a sweetener, and a fat.

17. The lipstick of claim 16, further comprising a moisturizer.

18. The lipstick of claim 16, further comprising an oil.

19. The lipstick of claim 16, further comprising a filler.

20. An edible candy nail polish comprising a sweetener, a solvent, and an edible shellac.

21. The nail polish of claim 20, further comprising a coloring.

22. The nail polish of claim 21, further comprising a flavoring.

23. The nail polish of claim 20, wherein the solvent comprises water.
